(19) United States
(12) Patent Application Publication
(10) Pub. No.: US 2006/0184155 A1
(43) Pub. Date: Aug. 17, 2006

(54) Manipulator and end effector for catheter manufacture

(76) Inventor: Rafael Fernandez-Sein, San German, PR (US)

Correspondence Address: Eugenio J. Torres;Ferramar Building Suite 1, 1060 Ashford Avenue, San Juan, PR, 00907, US

(21) Appl. No.: 11/026,688

(22) Filed: Dec. 31, 2004

(51) Int. Cl. A61M 25/00

(52) U.S. Cl. 604/526

(57) ABSTRACT

Manipulator end effector apparatus equipped with sensing capabilities attached to the manipulator arm for location of tubing or filamentary bodies is provided. The end effector picks up tubing with vacuum assisted bellows and presents it to the open jaws of two hooks in between a pair of rubber covered wheels. The rubber covered wheels rotate to advance the tubing to a work piece for insertion, assembly, testing, or inspection. If the tubing is clamped at the distal end, transfer to another section of the working surface is accomplished by simple sliding through the hooks while suitable guided by a robot arm. In another separate arrangement, a static manipulator fixed to the working surface is used to move the tubing longitudinally, or rotated on its axis for similar manufacturing or inspection steps.

### STATEMENT REGARDING FEDERALLY SPONSORED RESEARCH AND DEVELOPMENT

N/A

### RELATED APPLICATIONS

N/A

### BACKGROUND OF THE INVENTION

1. Field of the Invention

The present invention relates generally to the manufacture, assembly, and termination of catheters and cables for use or as a part of medical devices or for the interconnection of electrical or electronic devices.

2. Discussion of the Background

Catheters are flexible medical devices that are inserted into a human or animal body to perform different procedures, such as exploration, sampling, surgery, expansion, and connection of devices. Catheters must perform under conflicting considerations: they must be flexible enough to pass through veins and arteries, but stiff enough not to collapse under pressure. Catheters may also contain wires and fluid conductors that must be isolated from the subject body. Certain cables also perform tasks similar to those of catheters, such as connecting pacemakers with the heart of the patients.

During manufacture, catheter or cable parts may be placed in bundles to facilitate the movements of the parts through the different manufacturing steps. While this presents no problem to a human operator, the automation of these processes requires a higher degree of sensor integration and coordination.

Catheters, tubing, cables, wires, and filamentary materials are notoriously difficult to handle, and have been the focus of much research. See for example Robot Manipulation of Deformable Objects, Dominik Henrich and Heinz Wörn editors, ISBN, 1-85233-250-6 Springer Verlag London Limited 2000; and Mechanics of Robotic Manipulation by Matthew T. Mason, ISBN 0-262-13396-2 The MIT Press, Cambridge, Mass. 2001. However, no systematic solution to the problem of threading catheters has been found in the literature.

Some methods related to the problem at hand are found in the following patents, which are incorporated herein by reference:
A. U.S. Pat. No. 4,492,847 to Ichiro Masaki et al. for a Manipulator Welding Apparatus for Weld Slam Tracking. This patent teaches the general approach for a machine to traverse a taut welding path guided by image sensed by an optic fiber mounted on the moving robot arm.
B. U.S. Pat. No. 5,033,809 to Nobuo Shiga, for an Apparatus for Manufacturing an Optical Transmission Module. This patent discloses the apparatus for the positioning of an optical fiber while it is joined to an optical transmission module.
C. U.S. Pat. No. 6,275,748 B1 to Paul Bachi and Paul S. Filipski, for a Robot Arm with Specimen and Edge Gripping End Effector. This patent teaches the construction of a specialized gripping end effector that uses fiber optic light transmission sensors to locate the edge of rigid semiconductor wafers.
D. U.S. Pat. No. 6,453,214 to Paul Bacchi, and Paul S. Filipski, for a Method of Using a Specimen Sensing End Effector to Align a Robot Arm With a Specimen Stored or in a Container. This patent teaches the method of using the end effector presented in '748 issued to the same inventors.
E. U.S. Pat. No. 5,022,952 to Milo M. Vaniglia for a Fiber Placement Machine. This patent discloses the machinery for laying multiple fibers or filamentary material in a resin matrix to form composite material parts.
F. U.S. Pat. No. 6,736,156 to Scott A. Beals and Ronald D. Hammer for a Method and System for Installing Cable in Pressurized Pipelines. This patent teaches the use of belts on rollers to push optic fiber through a sealed housing into a pressurized pipeline.
G. U.S. Pat. No. 6,021,244 to Ronald Simpson, for Fiber Optic Stitching Process and Apparatus. This patent presents the method and apparatus by which an optic fiber is stitched together by a tweezers tipped gripper mounted on a Cartesian robotic assembly which interacts with another Cartesian robotic movement.

While these devices or methods in the prior art fulfill their respective, particular objectives and requirements, none of them, however, provide for end effectors or manipulators of tubing or filamentary materials, all eminently deformable bodies, to aid in the manufacture of catheters or cables for medical devices. In this regard, the present invention substantially fulfills this need.

### SUMMARY OF THE INVENTION

It is an object of the present invention to provide an apparatus to pick and grasp a section of tubing or filamentary material with the purpose of taking it through several steps in the manufacture of a catheter or cable device.

It is a further object of the present invention to provide the means to grasp a section of tubing or filamentary material with rotating and force controlled means to accomplish the movement or placement of said tubing for the particular manufacturing step.

It is another object of the present invention to measure the amount of rotation or movement of the grasping means for the tubing or filamentary material.

It is another object of the present invention to provide a sensor to determine the position and lay of the section of tubing or filamentary material to be picked up and grasped.

Yet another object of this invention is to provide a static or fixed in place manipulator apparatus to provide for the axial and longitudinal movement of tubing or filamentary objects and assist in the different steps in catheter or cable manufacture.

Further objects and advantages are to provide the robot arm end effector of this invention a sensor to quickly determine the position of tubing or filamentary material by sensing the distance to the from the effector head to the table. When the sensor finds a hump, tracking back and forth over this hump determines the position and lay of the tubing. Once the tubing is located, the end effector lowers two or more vacuum assisted bellows with a multiplicity of holes which must be properly aligned with the tubing for the pick up to take place. Sensing of the amount of vacuum indicates when alignment has been achieved. The vacuum bellows can then be retracted so that the tubing pass through the opened jaws of one or more hooks and between two rubber-covered wheels. Operating the rubber-covered wheels while the tubing rests on the hooks allows for the tubing to be moved longitudinally or to be presented to a manufacturing device for termination or insertion of a fitting into the tubing.

In still another aspect and advantage of the present invention, the manipulator is used as a static device which is able to move the tubing or filamentary material longitudinally or to make it rotate on its long axis. This way the manipulator can be during insertion, assembly, testing, or inspection manufacturing steps.

When the word “invention” is used in this specification, the word “invention” includes “inventions”, that is, the plural of “invention”. By stating “invention”, the Applicant does not in any way admit that the present application does not include more than one patentably and non-obviously distinct invention and Applicant maintains that the present application may include more than one patentably and non-obviously distinct invention.

Further, the purpose of the accompanying abstract is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

### DESCRIPTION OF THE DRAWINGS

FIG. 1 depicts the general layout and environment in which the invention is operated. The sample task depicted is to transfer a piece tubing from a bundle to a tray with v shaped grooves.

FIG. 2 shows a side view of the movable end effector with all its constituent components.

FIG. 3 is a side and cut away view of the slider mechanism that supports the vacuum suction tip.

FIG. 4 is a side view of the vacuum suction tip.

FIG. 5 is a bottom view of the vacuum suction tip.

FIG. 6 is a side view of gripper with a tweezers tip and a hook for the tubing or filamentary material to slide through.

FIG. 7 is a side view of the laser distance detector.

FIG. 8 is a profile of a height scan obtained from the distance sensor.

FIG. 9 is a general view of the static tubing or filamentary material manipulator.

FIG. 10 is a side view of the mechanism for the radial rotation of catheters or filamentary bodies.

FIG. 11 is a side view of the mechanism for the longitudinal advancement of catheters or filamentary bodies.

### DESCRIPTION OF THE PREFERRED EMBODIMENT

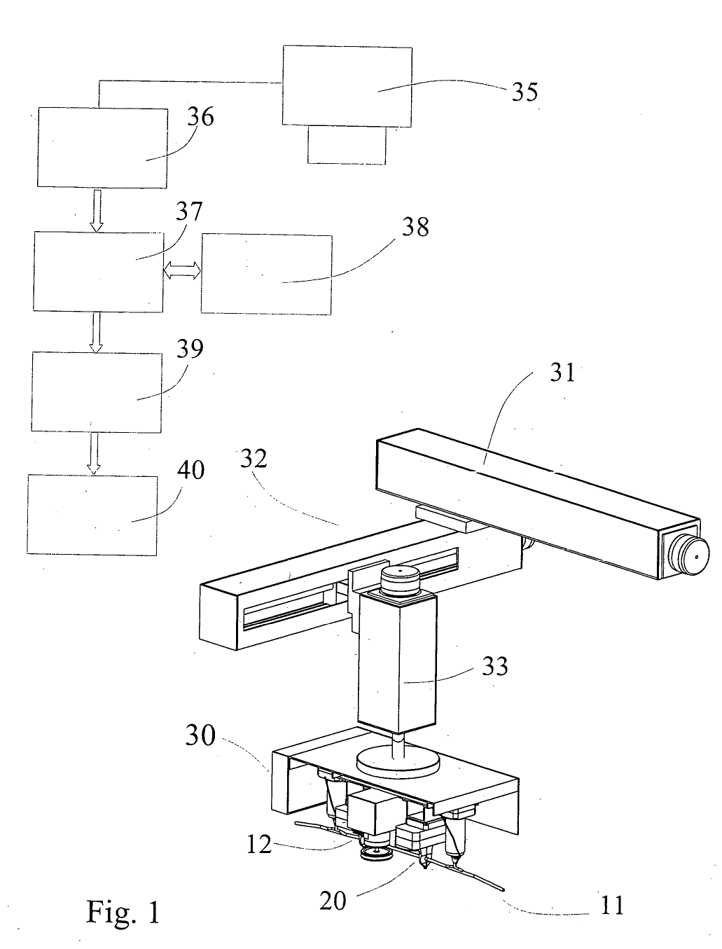

Referring now to the drawings, wherein like reference numerals designate the corresponding structure, part, or element, as the case may be, throughout the views, and particularly to FIG. 1, the end effector is moved in three axes by sliders 31,32,33, which form a Cartesian robotic arrangement. The entire scene is supervised by a digital camera 35 which feeds an image processor 36 which in turn delivers an image to a computer 37. From this image a rough position and lay of the tubing or filamentary body which is to be processed is derived. A plurality of position, force, and torque sensors report through sensor input module 38, transferring the data to said computer. The computer controls the entire operation through controllers 39 and drivers 40.

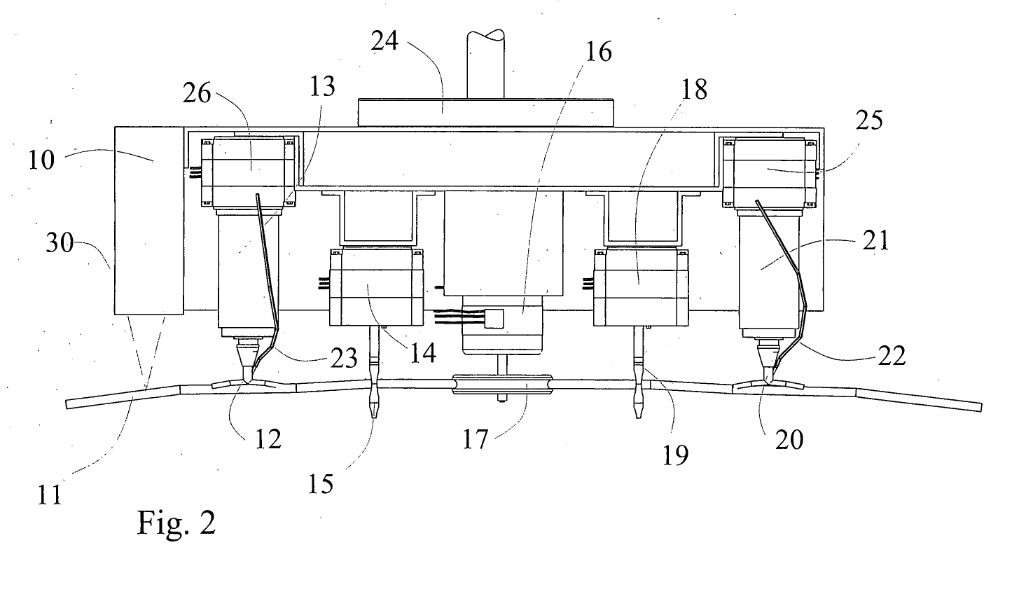

Referring to FIG. 1, the end effector is moved back and forth in a scanning motion, and with its laser distance sensor 30 determines a profile in three dimensions to ascertain the exact position and lay of the tubing or filamentary body. With reference to FIG. 2, the end effector may lower its suction probes 12 and 20 to lift filament 11 via linear actuators 13 and 21, electric motors 25 and 26, and controlled vacuum connections 22 and 23. Hooks 15 and 19 are opened by gripper movements 14 and 18 and then closed when said tubing is lifted onto the hook eyes. The hooks are closed and the tubing is now in place to be moved longitudinally by rubber wheels 17 and electric motors 16 mounted on slider base 10. The entire assembly is connected to Cartesian transport mechanism via 24.

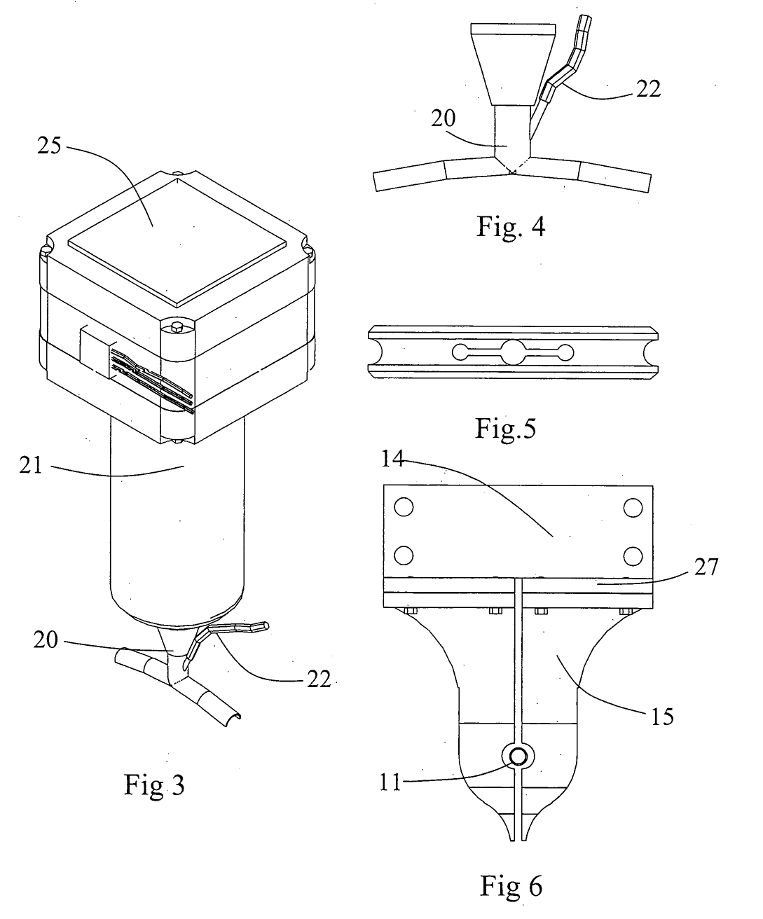

FIG. 3 shows a side view of the vacuum suction tip 20 and the linear actuator movement 29 which allows to be lowered or raised on command. FIG. 4 shows a side view of the suction tip and the connection to a controlled vacuum line 22. FIG. 5 shows a bottom view of the vacuum suction tip. FIG. 6 presents a side view of the gripper movement 14 with jaws 27 and pincers-hook combination 15 with filament 11 resting on hook.

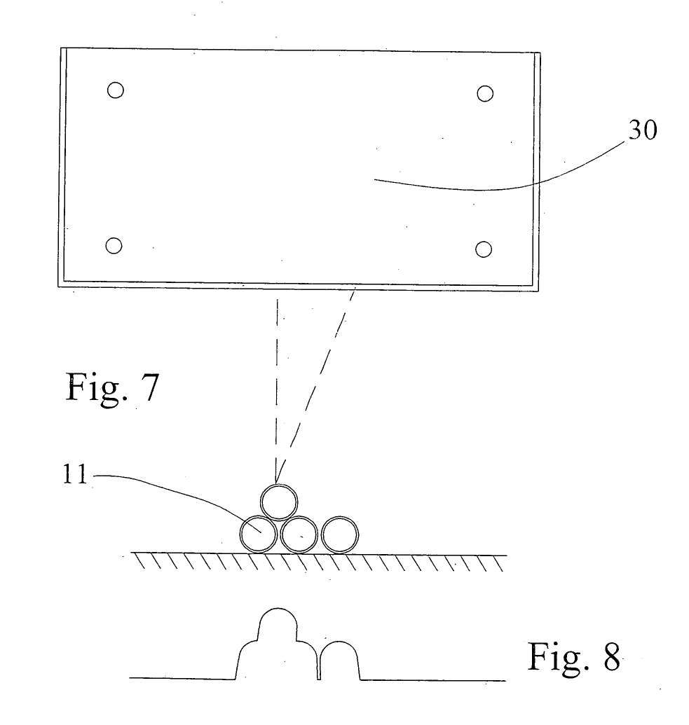

FIG. 7 is a side view of the diode laser distance measurement means 30 which is used to scan back and forth across the pile of filaments to processed. The distance detector determines a cross section of the pile as shown on FIG. 8. This information is used to determine which filament is on top of the pile.

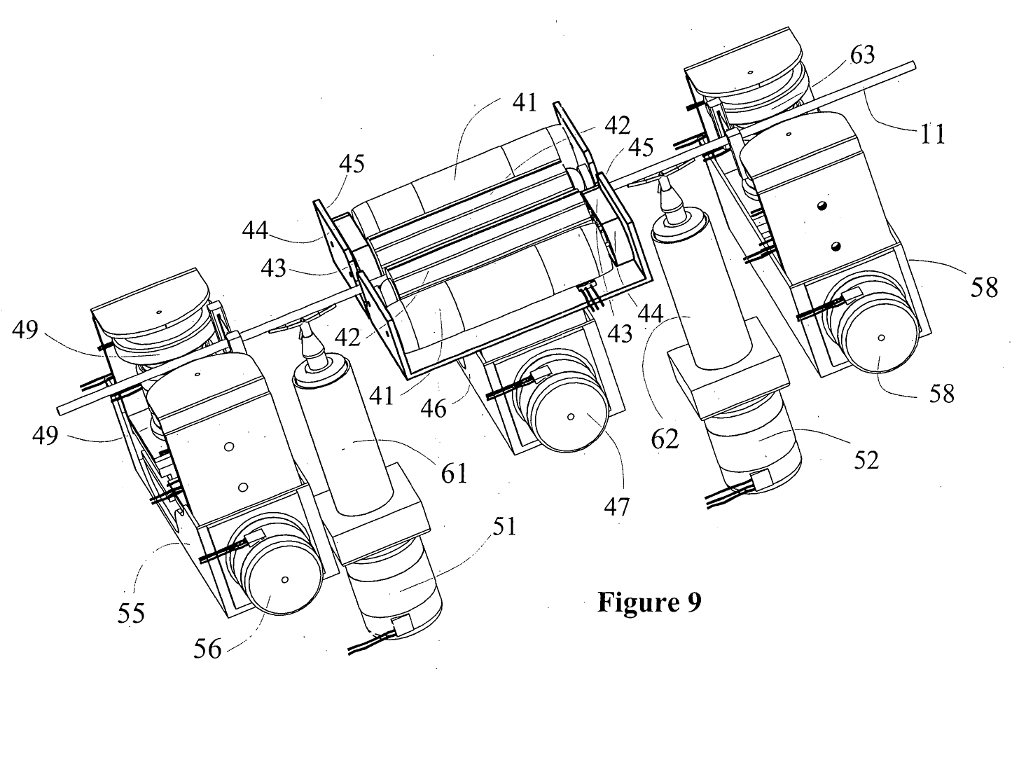

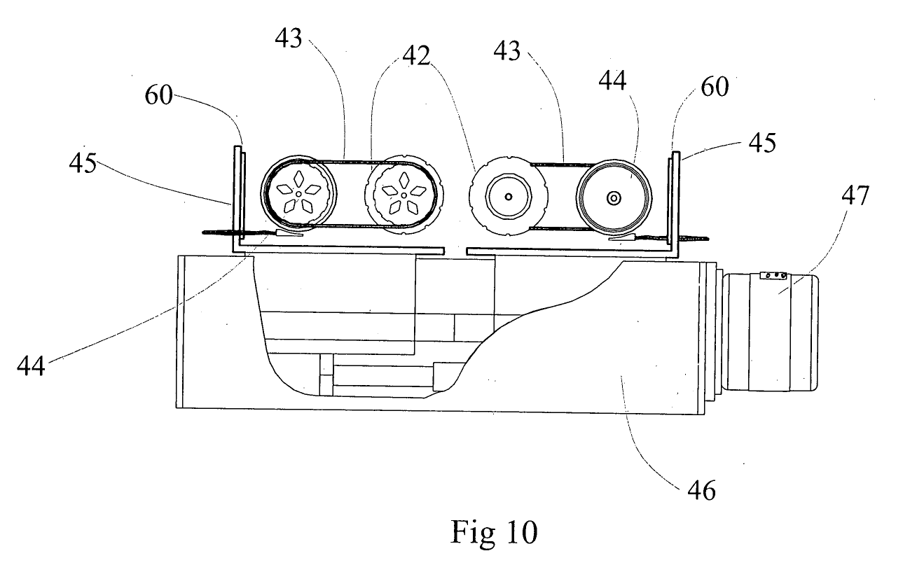

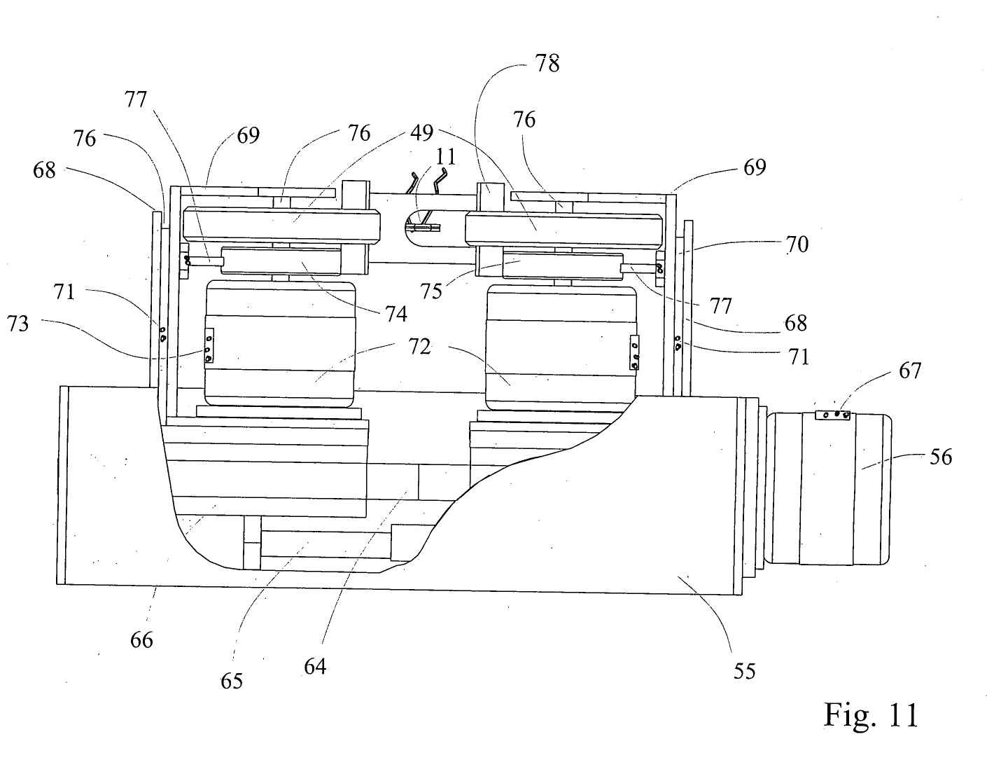

FIG. 9 presents a general view of the static manipulator assembly. Slider base 46 and controlled motor 47 provide opening and closing means to motors 41 connected to through sprocket 44 and chain 43 to rollers 42. Motor and roller assembly are slideably connected to slider base 45 which in turn is attached to slider base 45. Force sensor 60 measures the gripping force applied to filament 11. Linear actuators 61 and 62 powered by controlled motors 51 and 52 elevate vacuum tips 53 and 54. Referring to FIG. 11, rollers 49 and 63 and motors are slidably mounted on slider bases 55 and 57 are provided with opening and closing means by motors 56 and 58. Force sensors 64 and 65 and position sensors 66 and 67 provide feedback to control computer 37.

All of the patents recited herein, and in the Declaration attached hereto, if any, are hereby incorporated by reference as if set forth in their entirety herein. The details in such patents may be considered to be incorporable at Applicant's option, into the claims during prosecution as further limitations in the claims to patentably distinguish any amended claims from any applied prior art. The components disclosed in the various patents, patent applications, and publications, disclosed or incorporated by reference herein may be used in the embodiments of the present invention, as well as equivalents thereof.

All, or substantially all, of the components and methods of the various embodiments may be used with at least one embodiment or all of the embodiments, if more than one embodiment is described herein.

In the claims, means-plus-function clauses, if any, are intended to cover the structures described herein as performing the recited function and not only structural equivalents but also equivalent structures.

What is claimed is:

1. A catheter or filamentary material manipulator, comprising:
a. A plurality of slider bases;
b. A motor and roller assembly slideably connected to one of said slider bases;
c. at least three sliders forming a Cartesian robotic arrangement to move the end efector in three axes;
d. A plurality of gripper jaws equipped with force and position means, to enable longitudinal movement of said catheter or filamentary material;
e. A plurality of gripper movements for the controlled opening and closing of said jaws;
f. A plurality of distance, position, and torque sensors wherein said sensors are able to determine the position and lay of the catheter or filamentary material to be manipulated;
g. A plurality of force sensors wherein said force sensors are capable of measuring the gripping force applied to the catheter or filament;
h. A plurality of controlled vacuum suction tips equipped with longitudinal positioning means to pick-up the catheter or filamentary material;
i. A plurality of hooks said hooks being opened by said gripper movements and then closed once the catheter or filament is lifted;
j. A plurality of electric motors;
k. A plurality of electric motor controllers;
l. A plurality of drivers;
m. a camera suitable to feed images to an image processor means;
n. an image processor means suitable to feed images to a computer based controller;
o. A sensor input module wherein said plurality of distance, position, force and torque sensors report information to said module which in turns transfers processed data to said computer based controller;
p. A computer based controller with stored program means to control the entire operation of the manipulator through said controllers and drivers;
q. A plurality of wheels with rotation and torque controlled means to enable the longitudinal advancement of said tubing or filamentary material.

2. The catheter or filamentary material manipulator of claim 1, further comprising a plurality gripper or positioning stages with force and position control means.

3. The catheter or filamentary material manipulator of claim 1, wherein said plurality of wheels further enable the axial rotation of said catheter or filamentary material.

4. The catheter or filamentary material manipulator of claim 1, said electric motors having torque and rotation control means.

5. The catheter or filamentary material manipulator of claim 1, said vacuum tips means being further enabled to fix or constrain the catheter or filament while it is manipulated.
